(19) United States
(12) Patent Application Publication
(10) Pub. No.: US 2024/0245442 A1
(43) Pub. Date: Jul. 25, 2024

(54) ABLATION SYSTEM HAVING PULMONARY VEIN PRESSURE SENSING AND DISPLAY

(71) Applicant: Boston Scientific Scimed, Inc., Maple Grove, MN (US)

(72) Inventor: Chadi Harmouche, Saint-Laurent (CA)

(21) Appl. No.: 18/410,635

(22) Filed: Jan. 11, 2024

Related U.S. Application Data
(60) Provisional application No. 63/440,219, filed on Jan. 20, 2023.

(51) Int. Cl. A61B 18/02; A61B 18/00

(52) U.S. Cl. CPC A61B 18/02; A61B 2018/0022; A61B 2018/00744; A61B 2018/00916; A61B 2018/0212; A61B 2018/0262

(57) ABSTRACT

A cryogenic balloon catheter system for treating a condition in a patient includes a balloon catheter including a shaft, an expandable balloon attached to a distal portion of the shaft, and a pressure sensor coupled to the balloon catheter at a location distal to the expandable balloon. The system further includes a pump coupled to and configured to inflate the expandable balloon with a fluid, a graphical display configured to display a complication of data relating to the balloon catheter and a control system adapted to selectively inflate the expandable balloon and detect and display on the graphical display a pressure waveform sensed by the pressure sensor.

### CROSS-REFERENCE TO RELATED APPLICATIONS

This application claims the benefit of U.S. Provisional Application No. 63/440,219, entitled “ABLATION SYSTEM HAVING PULMONARY VEIN PRESSURE SENSING AND DISPLAY,” filed Jan. 20, 2023.

### TECHNICAL FIELD

The present disclosure relates to medical devices and methods for performing cryoablation procedures using a cryoablation catheter. More specifically, the disclosure relates to devices and methods for determining vessel occlusion by measuring and displaying pressure.

### BACKGROUND

Cardiac arrhythmias involve an abnormality in the electrical conduction of the heart and are a leading cause of stroke, heart disease, and sudden cardiac death. Treatment options for patients with arrhythmias include medications and use of medical devices, including implantable devices and catheter ablation of cardiac tissue. Catheter ablation involves delivering ablative energy to tissue inside the heart to block aberrant electrical activity from depolarizing heart muscle cells out of synchrony with the heart's normal conduction pattern. An ablation procedure may be performed by positioning the tip of an energy delivery catheter adjacent to targeted tissue in or near the heart.

The energy delivery component of the system is typically at or near the most distal (i.e., the furthest from the user or operator) portion of the catheter, and often at the tip of the catheter. Various forms of energy can be used to ablate diseased heart tissue. These can include, for example, radio frequency (RF), cryogenics, ultrasound, electrical fields, and laser energy. During a cryoablation procedure, with the aid of a guide wire, the distal tip of the catheter is positioned adjacent to targeted cardiac tissue, at which time energy is delivered to create tissue necrosis, rendering the ablated tissue incapable of conducting electrical signals. The dose of the energy delivered is an important factor in increasing the likelihood that the treated tissue is rendered permanently incapable of conduction.

Atrial fibrillation (AF) is one of the common arrhythmias treated using catheter ablation. In the earliest stages of the disease, paroxysmal AF, the treatment strategy may involve isolating the pulmonary veins from the left atrial chamber. One catheter procedure to treat AF is known as “balloon cryotherapy,” which offers ease of use, shorter procedure times and improved patient outcomes.

A goal of balloon cryotherapy is to isolate one or more pulmonary veins of the patient by creating circumferential transmural lesions around an ostium of the pulmonary vein being treated. During balloon cryotherapy, one or more cryogenic balloons are placed in the left atrium and positioned against the ostium of the pulmonary vein to occlude blood flow from the pulmonary veins into the left atrium. With the cryogenic balloons appropriately positioned to occlude the targeted tissue, a cryogenic fluid (e.g., nitrous oxide) is delivered under pressure to an interior of the one or more cryogenic balloons. The cryogenic fluid causes necrosis of the targeted tissue, thereby rendering the ablated tissue incapable of conducting electrical signals.

Pulmonary vein occlusion is typically a good indicator of complete circumferential contact between the balloon and ostium of the pulmonary vein, which facilitates optimal heat transfer during the ablation procedure. Such occlusion is often determined using contrast agents or fluoroscopy.

### SUMMARY

In Example 1, a cryogenic balloon catheter system includes a balloon catheter including a shaft, an expandable balloon attached to a distal portion of the shaft, and a pressure sensor coupled to the balloon catheter at a location distal to the expandable balloon; a pump coupled to and configured to inflate the expandable balloon with a fluid; a graphical display configured to display a complication of data relating to the balloon catheter; and a control system adapted to (a) selectively inflate the expandable balloon and (b) detect and display on the graphical display a pressure waveform sensed by the pressure sensor.

Example 2 is the cryogenic balloon catheter system of Example 1 further comprising a fluid source and a fluid control system for controlling delivery of the fluid to an interior of the expandable balloon.

Example 3 is the cryogenic balloon catheter system of Examples 1 or 2, further comprising a handle assembly attached to the shaft and operable by a user to control the balloon catheter and a connection component adapted to communicatively couple with the control system.

Example 4 is the cryogenic balloon catheter system of any of Examples 1-3 wherein the balloon catheter further includes an injection tube for conveying the fluid along the balloon catheter to an interior of the expandable balloon.

Example 5 is the cryogenic balloon catheter system of any of Examples 1-4 wherein the balloon catheter further includes a guide component coupled to a distal end of the shaft and extending through an interior of the expandable balloon and distal to the expandable balloon.

Example 6 is the cryogenic balloon catheter system of Example 5 wherein the pressure sensor is coupled to the guide component.

Example 7 is the cryogenic balloon catheter system of Example 6 wherein the pressure sensor is integrated into the guide component.

Example 8 is the cryogenic balloon catheter system of any of Examples 1-7 wherein the pressure sensor is coupled to a distal portion of the expandable balloon.

Example 9 is the cryogenic balloon catheter system of any of Examples 2-8 wherein the expandable balloon includes an inner balloon disposed inside an outer balloon and further wherein the inner balloon is adapted to receive the fluid.

Example 10 is the cryogenic balloon catheter system of any of Examples 1-9 wherein the control system is configured to further display a plurality of operating parameters associated with the cryogenic balloon catheter system.

Example 11 is the cryogenic balloon catheter system of any of Examples 1-10 wherein the control system is configured to display the pressure waveform during an inflation procedure and, upon initiation of an ablation procedure, to replace the pressure waveform with a temperature display.

Example 12 is the cryogenic balloon catheter system of any of Examples 1-11 wherein the control system is configured to monitor the pressure waveform during an inflation procedure and determine whether an occlusion has occurred based on a change in the pressure waveform.

Example 13 is the cryogenic balloon catheter system of any of Examples 1-12 wherein the pressure waveform is based on a pulmonary vein pressure and the control system is configured to determine whether an occlusion has occurred based on a change in the pressure waveform.

Example 14 is the cryogenic balloon catheter system of Example 13 wherein the control system is further configured to monitor a temperature waveform during the ablation procedure.

Example 15 is the cryogenic balloon catheter system of Example 1 where in the pressure waveform is based on a pulmonary vein pressure.

In Example 16, a cryogenic balloon catheter system includes a balloon catheter including a shaft, an expandable balloon attached to a distal portion of the shaft, and a pressure sensor coupled to the balloon catheter at a location distal to the expandable balloon; a pump coupled to and configured to inflate the expandable balloon with a fluid; a graphical display configured to display a complication of data relating to the balloon catheter; and a control system adapted to (a) selectively inflate the expandable balloon and (b) detect and display on the graphical display a pressure waveform sensed by the pressure sensor.

Example 17 is the cryogenic balloon catheter system of Example 16 further comprising a fluid source and a fluid control system for controlling delivery of the fluid to an interior of the expandable balloon.

Example 18 is the cryogenic balloon catheter system of Example 16, further comprising a handle assembly attached to the shaft and operable by a user to control the balloon catheter and a connection component adapted to communicatively couple with the control system.

Example 19 is the cryogenic balloon catheter system of Example 16 wherein the balloon catheter further includes an injection tube for conveying the fluid along the balloon catheter to an interior of the expandable balloon.

Example 20 is the cryogenic balloon catheter system of Example 16 wherein the balloon catheter further includes a guide component coupled to a distal end of the shaft and extending through an interior of the expandable balloon and distal to the expandable balloon. Example 21 is the cryogenic balloon catheter system of Example 20 wherein the pressure sensor is coupled to the guide component.

Example 22 is the cryogenic balloon catheter system of Example 21 wherein the pressure sensor is integrated into the guide component.

Example 23 is the cryogenic balloon catheter system of Example 16 wherein the pressure sensor is coupled to a distal portion of the expandable balloon.

Example 24 is the cryogenic balloon catheter system of Example 17 wherein the expandable balloon includes an inner balloon disposed inside an outer balloon and further wherein the inner balloon is adapted to receive the fluid.

Example 25 is the cryogenic balloon catheter system of Example 16 wherein the control system is configured to further display a plurality of operating parameters associated with the cryogenic balloon catheter system.

Example 26 is the cryogenic balloon catheter system of Example 16 wherein the control system is configured to display the pressure waveform during an inflation procedure and, upon initiation of an ablation procedure, to replace the pressure waveform with a temperature display.

Example 27 is the cryogenic balloon catheter system of Example 16 wherein the control system is configured to monitor the pressure waveform during an inflation procedure and determine whether an occlusion has occurred based on a change in the pressure waveform.

Example 28 is the cryogenic balloon catheter system of Example 16 where in the pressure waveform is based on a pulmonary vein pressure.

In Example 29, a method of performing a cryoablation procedure on a heart of a patient includes advancing a balloon catheter to a target location adjacent the heart, the balloon catheter including a shaft, an expandable balloon attached to a distal portion of the shaft, and a pressure sensor coupled to the balloon catheter at a location distal to the expandable balloon, initiating an inflation procedure including delivering a cryogenic fluid to an interior of the expandable balloon and generating a pressure waveform sensed by the pressure sensor during the inflation procedure, generating a display including a panel showing at least a portion of the pressure waveform during the inflation procedure; and upon completion of the inflation procedure, replacing the panel showing the pressure waveform with a temperature panel.

Example 30 is the method of Example 29 further including determining an occlusion based on a change in the pressure waveform.

Example 31 is the method of Example 30 wherein the pressure waveform is based on the pulmonary vein pressure and the occlusion is based on the pulmonary vein.

Example 32 is the method of Example 29 further comprising, following completion of the inflation procedure, initiating an ablation procedure.

Example 33 is the method of Example 29 wherein the delivering step includes using a fluid source and a fluid control system for controlling delivery of the fluid to an interior of the expandable balloon.

Example 34 is the method of Example 29 wherein the balloon catheter further includes a guide component coupled to a distal end of the shaft and extending through an interior of the expandable balloon and distal to the expandable balloon.

Example 35 is the method of Example 34 wherein the pressure sensor is disposed within the pulmonary vein.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic side view illustration of a patient and of a cryogenic balloon catheter system, including a cryogenic balloon catheter assembly and a control and display system according to embodiments of the present invention.

FIG. 2 is a schematic view illustration of a portion of a patient's heart and a portion of the cryogenic balloon catheter system, including a pressure sensor, according to embodiments of the present invention.

FIG. 3 shows exemplary pressure waveforms during various phases of a cryogenic treatment procedure, according to embodiments of the present invention.

FIG. 4A shows an exemplary pressure waveform in the left atrium during prior to pulmonary vein occlusion, and FIG. 4B shows an exemplary pressure waveform in the pulmonary vein after occlusion of the ostium using the cryogenic balloon catheter assembly, according to embodiments of the present invention.

FIG. 5 is a flow chart showing a method of displaying pressure on the graphical display, according to embodiments of the present invention

FIG. 6 shows a graphical display having a variety of panels displaying information relevant to the procedure, including a pressure waveform panel, according to embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

The following detailed description is exemplary in nature and is not intended to limit the scope, applicability, or configuration of the invention in any way. Rather, the following description provides some practical illustrations for implementing exemplary embodiments of the present invention. Examples of constructions, materials, and/or dimensions are provided for selected elements. Those skilled in the art will recognize that many of the noted examples have a variety of suitable alternatives.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings. Embodiments of the present invention, for example, are described herein in the context of a cryogenic balloon catheter system, but the concepts are also applicable in other types of ablation systems, including for example radio-frequency (RF), ultrasound, electric field and laser ablation. The ablation system includes a pressure sensor for detecting pressure in the left atrium and/or the pulmonary artery. This pressure is then displayed to the user as an indicator of pulmonary vein occlusion.

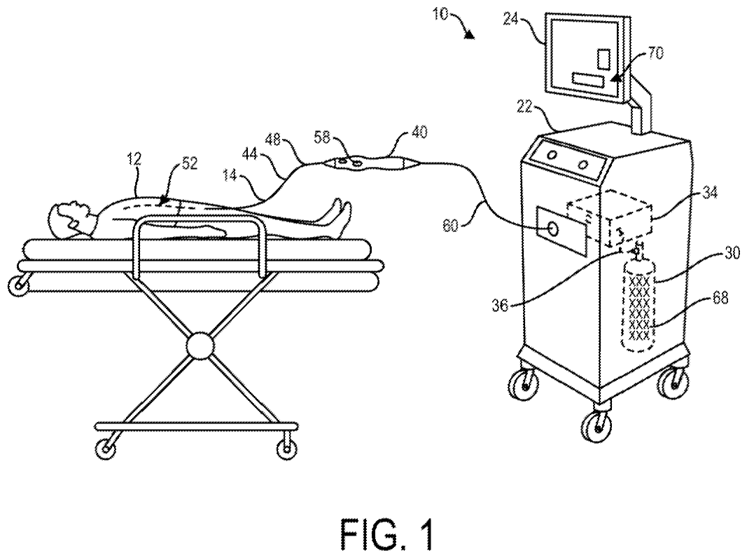

FIG. 1 is a simplified schematic side view illustration of an embodiment of a cryogenic balloon catheter system 10 for use with a patient 12, which can be a human being or an animal. Although the design of the cryogenic balloon catheter system 10 can be varied depending on the particular clinical needs of the patient 12, in the illustrated embodiment, the cryogenic balloon catheter system 10 can include one or more of a balloon catheter 14, a control console 22, a graphical display 24, and a control system 28 illustrated in phantom and disposed within the control console 22 in FIG. 1. In the illustrated embodiment, the control system 28 includes a fluid source 30 and a fluid control arrangement 34. In the various embodiments, the fluid control system 28 can include various conduits, valves and instrumentation configured to supply and withdraw a fluid to the active elements on the balloon catheter 14 as will be described in greater detail elsewhere herein. In the illustrated embodiment, the fluid source 30 is operably connected to the fluid control arrangement 34 by a conduit 36 (which may be in the form of a hose or tubing) configured to transfer fluid contained within the fluid source 30 to components making up the fluid control arrangement 34.

As further shown, the balloon catheter 14 includes a handle assembly 40, and a shaft 44 having a proximal end portion 48 connected to the handle assembly 40, and a distal end portion 52, shown disposed within the patient 12 in FIG. 1. As will be appreciated, the handle assembly 40 can include various components, such as the control element 58 in FIG. 1, that the user can manipulate to operate the balloon catheter 14. Also, in the embodiment illustrated in FIG. 1, an umbilical 60 operatively connects the handle assembly 40 and the active components of the balloon catheter 14 to the control console 22.

In various embodiments, the system 10 may also include additional components or alternative approaches to operatively connect the balloon catheter 14 to the control console 22. That is, the specific means of operatively connecting these elements is not critical the present disclosure, and so any suitable means can be employed.

It is understood that although FIG. 1 illustrates the structures of the cryogenic balloon catheter system 10 in a particular position, sequence and/or order, these structures can be located in any suitably different position, sequence and/or order than that illustrated in FIG. 1. It is also understood that the cryogenic balloon catheter system 10 can include fewer or additional components than those specifically illustrated and described herein.

In various embodiments, the fluid control system 28 is configured to monitor and control various processes of the ablation procedures performed with the cryogenic balloon catheter system 10. More specifically, the fluid control system 28 can monitor and control release and/or retrieval of a cooling fluid 68, e.g., a cryogenic fluid (shown schematically contained within the fluid source 30 in FIG. 1), to the balloon catheter 14, e.g., via fluid injection and fluid exhaust lines (not shown, but which may be disposed within the umbilical 60. The fluid control system 28 can also control various structures that are responsible for maintaining and/or adjusting a flow rate and/or pressure of the cryogenic fluid 68 that is released to the balloon catheter 14 during the cryoablation procedure. In such embodiments, the cryogenic balloon catheter system 10 delivers ablative energy in the form of cryogenic fluid 68 to cardiac tissue of the patient 12 to create tissue necrosis, rendering the ablated tissue incapable of conducting electrical signals. Additionally, in various embodiments, the fluid control system 28 can control activation and/or deactivation of one or more other processes of the balloon catheter 14, including for example measuring a pressure in the patient's pulmonary artery.

Further, or in the alternative, the fluid control system 28 can receive data and/or other information (hereinafter sometimes referred to as “sensor output”) from various structures within the cryogenic balloon catheter system 10. In some embodiments, the fluid control system 28 can receive, monitor, assimilate and/or integrate the sensor output, and/or any other data or information received from any structure within the cryogenic balloon catheter system 10 in order to control the operation of the balloon catheter 14. As provided herein, in various embodiments, the fluid control system 28 can initiate and/or terminate the flow of cryogenic fluid 68 to the balloon catheter 14 based on the sensor output.

As shown in FIG. 1, in certain embodiments, the fluid control system 28 can be positioned substantially within the control console 22. Alternatively, at least a portion of the fluid control system 28 can be positioned in one or more other locations within the cryogenic balloon catheter system 10, e.g., within the handle assembly 40.

The fluid source 16 contains the cryogenic fluid 68, which is delivered to and from the balloon catheter 14 with or without input from the fluid control system 28 during a cryoablation procedure. Once the ablation procedure has initiated, the cryogenic fluid 68 can be delivered and the resulting gas, after a phase change, can be retrieved from the balloon catheter 14, and can either be vented or otherwise discarded as exhaust. Additionally, the type of cryogenic fluid 68 that is used during the cryoablation procedure can vary. In one non-exclusive embodiment, the cryogenic fluid 68 can include liquid nitrous oxide. However, any other suitable cryogenic fluid 68 can be used. For example, in one non-exclusive alternative embodiment, the cryogenic fluid 68 can include liquid nitrogen.

The design of the balloon catheter 14 can be varied to suit the specific design requirements of the cryogenic balloon catheter system 10. As shown, the balloon catheter 14 is inserted into the body of the patient 12 during the cryoablation procedure. The handle assembly 40 can be handled and used by the operator to operate, position and control the balloon catheter 14. The design and specific features of the handle assembly 40 can vary to suit the design requirements of the cryogenic balloon catheter system 10. In the embodiment illustrated in FIG. 1, the handle assembly 40 is separate from, but in electrical and/or fluid communication with the fluid control system 28, the fluid source 16, and the graphical display 24. In some embodiments, the handle assembly 40 can integrate and/or include at least a portion of the fluid control system 28 within an interior of the handle assembly 40. It is understood that the handle assembly 40 can include fewer or additional components than those specifically illustrated and described herein. Additionally, in certain embodiments, the handle assembly 40 can include circuitry (not shown in FIG. 1) that can include at least a portion of the fluid control system 28. Alternatively, the circuitry can transmit electrical signals such as the sensor output, or otherwise provide data to the fluid control system 28 as described herein. In one embodiment, the circuitry can include a printed circuit board having one or more integrated circuits, or any other suitable circuitry.

Still further, in certain embodiments, the handle assembly 40 can be used by the operator to initiate and/or terminate the cryoablation process, e.g., to start the flow of the cryogenic fluid 68 to the balloon catheter 14 in order to ablate certain targeted heart tissue of the patient 12.

In the embodiment illustrated in FIG. 1, the control console 22 includes at least a portion of the fluid control system 28, the fluid source 16, and the graphical display 24. However, in alternative embodiments, the control console 22 can contain additional structures not shown or described herein. Still alternatively, the control console 22 may not include various structures that are illustrated within the control console 22 in FIG. 1. For example, in certain non-exclusive alternative embodiments, the control console 22 does not include the graphical display 24.

During cryoablation procedures, the balloon catheter 14 and the control console 22 must be mechanically connected to allow the flow of cryogenic fluid 68 from the control console 22 to the balloon catheter 14 and back to the control console 22. Generally, during the application of ablative energy, the cryogenic fluid 68 flows in a liquid phase to the balloon catheter 14. The cryogenic fluid 68 then undergoes a phase change and returns to the control console 22 as exhaust in a gaseous phase.

In various embodiments, the graphical display 24 is electrically connected to the fluid control system 28. Additionally, the graphical display 24 provides the operator of the cryogenic balloon catheter system 10 with information that can be used before, during and after the cryoablation procedure. For example, the graphical display 24 can provide the operator with information based on the sensor output, and any other relevant information that can be used before, during and after the cryoablation procedure. The specifics of the graphical display 24 can vary depending upon the design requirements of the cryogenic balloon catheter system 10, or the specific needs, specifications and/or desires of the operator.

In one embodiment, the graphical display 24 can provide static visual data and/or information to the operator via various frames or other representations (depicted as element 70 in FIG. 1). In addition, or in the alternative, the graphical display 24 can provide dynamic visual data and/or information to the operator, such as video data or any other data that changes over time, e.g., during an ablation procedure. Further, in various embodiments, the graphical display 24 can include one or more colors, different sizes, varying brightness, etc., that may act as alerts to the operator. Additionally, or in the alternative, the graphical display 24 can provide audio data or information to the operator.

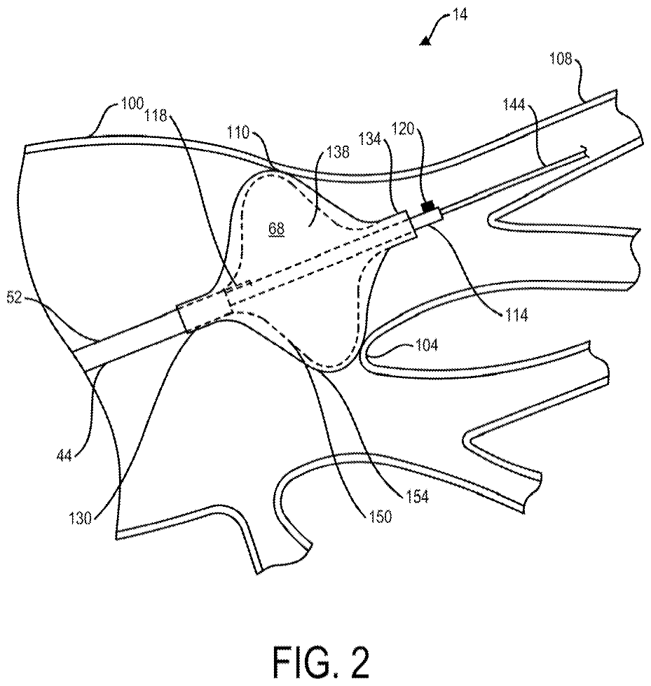

FIG. 2 is a schematic illustration of the distal end portion 52 of the balloon catheter 14 positioned within a selected anatomical region of the patient 12, in this case, a left atrium 100 adjacent to an ostium 104 of a pulmonary vein 108, such as when the system 10 is used in a pulmonary vein isolation (PVI) procedure to terminate an atrial fibrillation. In the illustrated embodiment, the balloon catheter 14 includes an expandable balloon 110, a guide catheter 114 (including a lumen) and an injection tube 118. The balloon catheter 14 further includes a pressure sensor 120. The guide catheter 114 includes a lumen adapted to receive and slide along a guide device such as, for example, a guidewire or a stylet.

In the illustrated embodiment, the pressure sensor 120 is coupled to an outside of the shaft the guide catheter 114. In other embodiments, the pressure sensor 120 is integrated into the guide catheter 114, and in various embodiments, the pressure sensor 120 located inside the lumen of the guide catheter 114. In other embodiments, the pressure sensor 120 is coupled to (or integrated into) a distal portion of the expandable balloon 110. In some embodiments, the pressure sensor 120 is coupled to (or integrated into) a guidewire or stylet. In various embodiment, the balloon catheter 14 includes more than one pressure sensor 120. As shown, when the balloon catheter 14 is positioned in the patient 12, the pressure sensor 120 is disposed within the pulmonary vein 108 of the patient 12. The pressure sensor 120 is operatively coupled to the control system 28, such that the control system may measure and determine a pressure of the patient's blood at the location of the sensor. According to various embodiments, the control system 28 is configured to detect the presence of the pressure sensor 120 and control operation of the system 10, based on the presence (or absence) of the sensor.

As shown, the balloon 110 has a proximal end 130 and an opposite distal end 134, the balloon 10 and defines an internal space 138 (i.e., an interior) that creates a cryo-chamber during a cryoablation procedure. In the illustrated embodiment, the proximal end 130 of the balloon 110 is attached to the distal end portion 52 of the shaft 44, and the distal end 134 of the balloon 110 is attached to the guide catheter 114 near the distal end thereof. In the illustrated embodiment, the injection tube 118 is disposed within and extends from the shaft 44, and terminates within and is open to the internal space 138. The injection tube 118 is operable to deliver the cryogenic fluid 68 to the internal space 138.

Although not shown in FIG. 2, the balloon catheter 14 also includes an exhaust lumen within the shaft 44 and open to the internal space 138. The exhaust lumen is operable to facilitate evacuation of the cryogenic fluid 68 from the internal space 138, and also to facilitate inflation of the balloon 110 as will be explained in further detail herein.

In various embodiments, the guide component may be slidable relative to the shaft 44 to facilitate expansion and subsequent collapse of the balloon 110 in use. However, the particular construction of the balloon 110 and guide catheter 114 is not critical to the present disclosure, and so other configurations may be used within the scope of the various embodiments.

For illustration purposes, an instrument 144 is shown extending through and beyond the guide component and into the pulmonary vein 108. As the skilled artisan will appreciate, the instrument 144 may be a guidewire, mapping wire or catheter, anchoring wire, or other medical device useful to facilitate the particular cryo-therapy procedure. In some embodiments, the pressure sensor 120 is coupled to (or integrated into) the instrument 144. The instrument 144, however, is optional and is not critical to the embodiments disclosed herein.

In the embodiment of FIG. 2, the balloon 110 is a dual-balloon construction including an inner balloon 150 and an outer balloon 154. The balloons 150, 154 are configured such that the inner balloon 150 receives the cryogenic fluid 68 (illustrated in FIG. 1), and the outer balloon 154 surrounds the inner balloon 150. The outer balloon 154 acts as part of a safety system to capture the cryogenic fluid 68 in the event of a leak from the inner balloon 150. It is understood that the balloon catheter 14 can include other structures as well. However, for the sake of clarity, these other structures have been omitted from the figures. Additionally, it is further appreciated that in some alternative embodiments, the balloon catheter 14 includes only a single balloon.

In the embodiment illustrated in FIG. 2, the balloon catheter 14 is positioned within the left atrium 100 of the patient 12. The guidewire 144 and guide catheter 114 are inserted into a pulmonary vein 108 of the patient 12, and the catheter shaft 44 and the balloons 150, 154 are moved along the guidewire 144 and/or the guide catheter 114 to be positioned near an ostium 104 of the pulmonary vein 108.

During use, the inner balloon 150 can be partially or fully inflated so that at least a portion of the inner balloon 150 expands against at least a portion of the outer balloon 154. Once the inner balloon 150 is sufficiently inflated, an outer surface of the outer balloon 154 can then be positioned to abut and/or substantially form a seal with the ostium 104 of the pulmonary vein 108 to be treated. In various embodiments, the pressure sensor 120 is used to measure pressure within the pulmonary vein during or after placement and inflation of the outer balloon 154. As further explained below, these pressure measurements can be used to confirm occlusion of the ostium 104 has been achieved. In certain embodiments, this inflation procedure is controlled and executed by the fluid control system 28.

The inner balloon 150 and the outer balloon 154 can be formed from any suitable materials. For example, in some embodiments, the inner balloon 150 can be formed from a sturdy material to better inhibit leaks of the cryogenic fluid 68 that is received therein, and the outer balloon 154 can be made from a relatively compliant material to ensure better contact and positioning between the outer balloon 154 and the pulmonary vein 108.

During balloon cryoablation procedures, prior to delivering the cryoablative energy, the operator can inflate the balloon using the cryogenic fluid 68 at a relatively high temperature (i.e., well above the temperature sufficient to ablate the target tissue). In this way, the operator can ensure sufficient balloon-tissue contact and vein occlusion before starting an ablation to increase probability of vein isolation. In addition, to minimize procedure time, it can be desirable to utilize the exhaust lumen of the balloon catheter 14 as a conduit for delivering the cryogenic fluid 68 to the internal space 138 during the inflation phase, i.e., due to its relatively large size compared to the injection tube 118. It is also desirable to maintain relatively close control over the inflation pressure during the cryoablation procedure. For example, a drop in the inflation pressure can result in partial deflation of the balloon 110 and diminishment of balloon tissue contact and vessel occlusion.

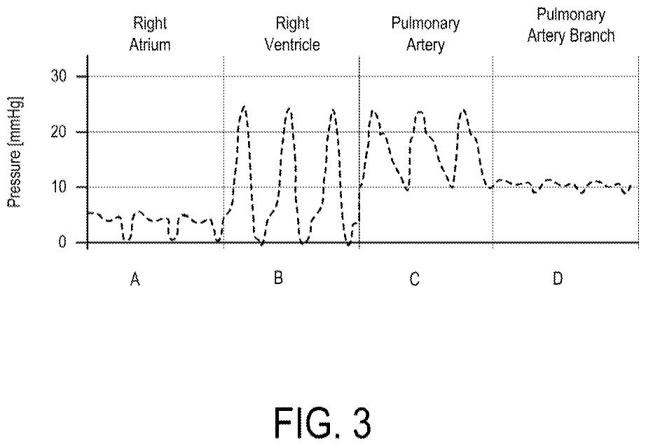

FIG. 3 shows exemplary pressure waveforms measured at various locations within the heart, according to embodiments of the present invention. As shown, the pressure waveform varies in amplitude and morphology in each location. FIG. 3, portion A, shows the pressure waveform in the right atrium. FIG. 3, portion B, shows the pressure waveform in the right ventricle. FIG. 3, portion C, shows the pressure waveform in the pulmonary artery. FIG. 3, portion D, shows the pressure waveform in the pulmonary artery branch. In various embodiments, any of these pressure waveforms can be used to detect occlusion of the pulmonary vein ostium, based on associated changes to the pressure waveform caused by such occlusion.

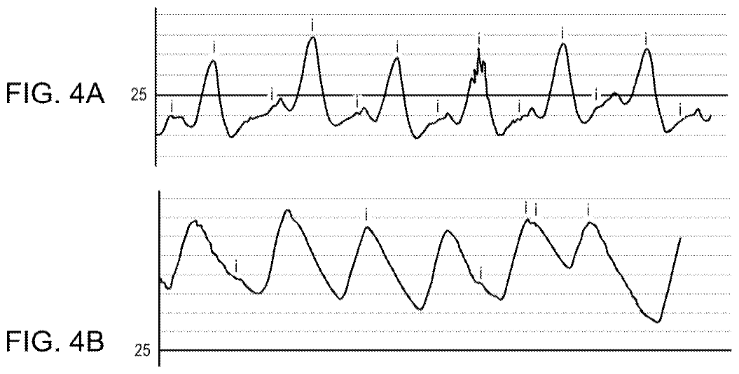

FIG. 4A shows a characteristic left atrium (or pulmonary vein) pressure trace prior to the balloon 150 occluding the pulmonary vein ostium 104. As shown, the waveform includes typical A-wave and V-wave morphologies, including the V-wave having a typical “isosceles triangle” morphology. As shown in FIG. 4B, when occlusion of the ostium 104 is achieved, the waveform changes substantially. During sinus rhythm, for example, the A-wave is lost. Also, the pressure amplitude increases substantially. Upon occlusion of the ostium 104, the pressure trace is essentially the trans-capillary pulmonary arterial pressure. As shown, the V-wave has a significantly more rapid rise and a more delayed downstroke. Thus, as exemplified by FIGS. 4A and 4B, it is possible to detect occlusion of the pulmonary vein ostium 104 by detecting changes in pulmonary vein (or left atrial) pressure. According to various embodiments, such pressure changes are detected by the pressure sensor 120 and communicated to the control system 28.

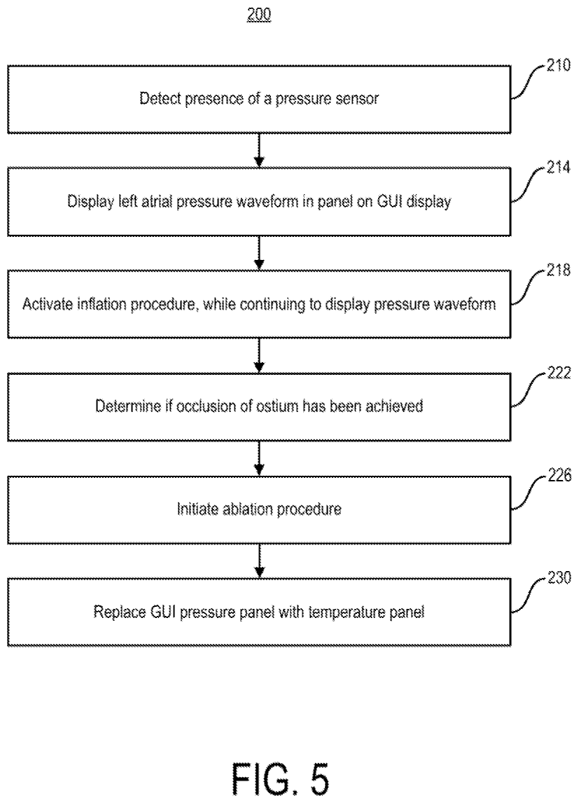

FIG. 5 is a flow chart showing a method 200 of operation of the cryogenic balloon catheter system 10, according to embodiments of the present invention. First, the control system 28 determines whether a pressure sensor 120 is present (block 210). If a pressure sensor is present, the control system 28 determines the pressure waveform measured by the pressure sensor 120 and displays the pressure waveform on the graphical display 24 (block 214). The control system 28 then activates an inflation procedure (as detailed above) to inflate the balloon, while also continuing to display the pressure waveform (block 218). In certain embodiment, the graphical display shows between two and twenty heartbeats in the pressure waveform panel. In other embodiments, the graphical display shows between four and ten heartbeats in the pressure waveform panel.

Once the user notes the changes to the pressure waveform associated with occlusion of the ostium 104 (as detailed above), the user inputs this determination using the control console 22 (block 222). The user then initiates the ablation procedure (block 226). In certain embodiments, the control system 28 detects the changes to the pressure waveform and automatically initiates the ablation procedure (blocks 222 and 226). Upon initiation of the ablation procedure, the inflation procedure is halted and the control system 28 replaces the display of the pressure waveform on the graphical display 24 with a display of a temperature panel, as is commonly displayed during an ablation procedure (block 230).

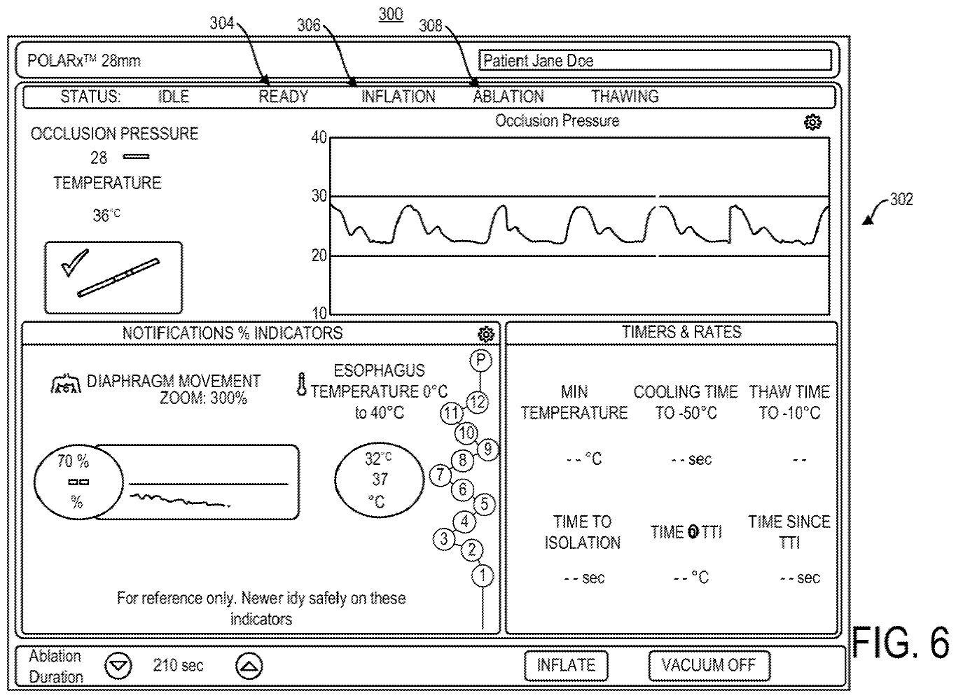

FIG. 6 shows a graphical display 300 having a variety of panels displaying information relevant to the procedure, including a pressure waveform panel 302, according to embodiments of the present invention. According to various embodiments, FIG. 6 represents the graphical display shown to the user during the certain phases of the ablation procedure. During an early phase, an indicator such as “READY” 304 may be shown on the graphical display 300 to indicate device initiation. As described above, during this phase, the system determines if a pressure sensor 120 is present and, if so, displays the pressure sensor waveform panel 302 on the graphical display. When a user initiates and inflation phase, an indicator such as “INFLATION” 306 may be shown on the graphical display, while it also continues to display the pressure waveform panel 302. As described above, the catheter is placed near the ostium 104 and the pressure inside the balloon is increased to cause inflation of the balloon. During this phase of the procedure, the graphical display 300 continues to show the pressure waveform panel 302. The user can then monitor this panel for the changes associated with occlusion of the pulmonary vein ostium as described above. Upon detection of these changes, the user can transition from the inflation phase to the ablation phase of the procedure. In connection with such a transition, an indicator such as “ABLATION” 308 may be shown on the graphical display. As explained above, when the operation phase transitions from inflation to ablation, the pressure waveform panel 302 is removed and replaced with a temperature panel (not shown).

According to various embodiments, the control system can detect the changes to the pressure waveform and provide an alert to the user. In certain embodiment, the controller can detect the changes and automatically stop the inflation of the balloon. In certain embodiments, the control system can switch the device from the inflation phase to the ablation phase.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A cryogenic balloon catheter system, comprising:
a balloon catheter including a shaft, an expandable balloon attached to a distal portion of the shaft, and a pressure sensor coupled to the balloon catheter at a location distal to the expandable balloon;
a pump coupled to and configured to inflate the expandable balloon with a fluid;
a graphical display configured to display a complication of data relating to the balloon catheter; and
a control system adapted to (a) selectively inflate the expandable balloon and (b) detect and display on the graphical display a pressure waveform sensed by the pressure sensor.

2. The cryogenic balloon catheter system of claim 1 further comprising a fluid source and a fluid control system for controlling delivery of the fluid to an interior of the expandable balloon.

3. The cryogenic balloon catheter system of claim 1, further comprising a handle assembly attached to the shaft and operable by a user to control the balloon catheter and a connection component adapted to communicatively couple with the control system.

4. The cryogenic balloon catheter system of claim 1 wherein the balloon catheter further includes an injection tube for conveying the fluid along the balloon catheter to an interior of the expandable balloon.

5. The cryogenic balloon catheter system of claim 1 wherein the balloon catheter further includes a guide component coupled to a distal end of the shaft and extending through an interior of the expandable balloon and distal to the expandable balloon.

6. The cryogenic balloon catheter system of claim 5 wherein the pressure sensor is coupled to the guide component.

7. The cryogenic balloon catheter system of claim 6 wherein the pressure sensor is integrated into the guide component.

8. The cryogenic balloon catheter system of claim 1 wherein the pressure sensor is coupled to a distal portion of the expandable balloon.

9. The cryogenic balloon catheter system of claim 2 wherein the expandable balloon includes an inner balloon disposed inside an outer balloon and further wherein the inner balloon is adapted to receive the fluid.

10. The cryogenic balloon catheter system of claim 1 wherein the control system is configured to further display a plurality of operating parameters associated with the cryogenic balloon catheter system.

11. The cryogenic balloon catheter system of claim 1 wherein the control system is configured to display the pressure waveform during an inflation procedure and, upon initiation of an ablation procedure, to replace the pressure waveform with a temperature display.

12. The cryogenic balloon catheter system of claim 1 wherein the control system is configured to monitor the pressure waveform during an inflation procedure and determine whether an occlusion has occurred based on a change in the pressure waveform.

13. The cryogenic balloon catheter system of claim 1 where in the pressure waveform is based on a pulmonary vein pressure.

14. A method of performing a cryoablation procedure on a heart of a patient, the method comprising:
advancing a balloon catheter to a target location adjacent the heart, the balloon catheter including a shaft, an expandable balloon attached to a distal portion of the shaft, and a pressure sensor coupled to the balloon catheter at a location distal to the expandable balloon;
initiating an inflation procedure including delivering a cryogenic fluid to an interior of the expandable balloon and generating a pressure waveform sensed by the pressure sensor during the inflation procedure;
generating a display including a panel showing at least a portion of the pressure waveform during the inflation procedure; and
upon completion of the inflation procedure, replacing the panel showing the pressure waveform with a temperature panel.

15. The method of claim 14 further including determining an occlusion based on a change in the pressure waveform.

16. The method of claim 15 wherein the pressure waveform is based on the pulmonary vein pressure and the occlusion is based on the pulmonary vein.

17. The method of claim 14 further comprising, following completion of the inflation procedure, initiating an ablation procedure.

18. The method of claim 14 wherein the delivering step includes using a fluid source and a fluid control system for controlling delivery of the fluid to an interior of the expandable balloon.

19. The method of claim 14 wherein the balloon catheter further includes a guide component coupled to a distal end of the shaft and extending through an interior of the expandable balloon and distal to the expandable balloon.

20. The method of claim 19 wherein the pressure sensor is disposed within the pulmonary vein.
